(19) United States
(12) Patent Application Publication
(10) Pub. No.: US 2018/0360776 A1
(43) Pub. Date: Dec. 20, 2018

(54) ANTI-OBESITY POTENTIAL OF GARCINOL

(71) Applicants: Muhammed Majeed, Edison, NJ (US); Kalyanam Nagabhushanam, East Windsor, NJ (US); Lakshmi Mundkur, Bangalore (IN)

(72) Inventors: Muhammed Majeed, Edison, NJ (US); Kalyanam Nagabhushanam, East Windsor, NJ (US); Lakshmi Mundkur, Bangalore (IN)

(21) Appl. No.: 16/007,212

(22) Filed: Jun. 13, 2018

Related U.S. Application Data
(60) Provisional application No. 62/519,948, filed on Jun. 15, 2017.
(60) Provisional application No. 62/523,611, filed on Jun. 22, 2017.

(51) Int. Cl. A61K 31/122; A61P 3/04; A61P 3/06

(52) U.S. Cl. CPC A61K 31/122; A61K 9/0053; A61P 3/06; A61P 3/04

(57) ABSTRACT

Disclosed are compositions containing garcinol for the therapeutic management of obesity. More specifically, the invention relates to the use of garcinol for a) maintaining energy balance in mammalian adipose cellular systems b) management of hypercholesterolemia and c) reducing weight gain in mammals. The modification of gut microbiota and the increase of beneficial microbe, Akkermansia muciniphila by garcinol are also disclosed.

### CROSS-REFERENCE TO RELATED PATENT APPLICATION

The present invention is a non-provisional application of the U.S. provisional patent application Nos. 62/519,949 filed on 15 Jun. 2017 and 62/523,611 filed on 22 Jun. 2017.

### BACKGROUND OF THE INVENTION

### Field of the Invention

The invention in general relates to compositions for weight management. Specifically the invention relates to compositions containing garcinol for the management of obesity, hypercholesterolemia and modification of gut microbiota.

### Description of Prior Art

Obesity is considered to be the leading health risk for the development of various disorders like hypertension, type 2 diabetes, heart disease, stroke, osteoarthritis, and mental illness. Globally, more than 1 in 10 individuals are obese and about 36% of American adults are obese (https://www.medicalnewstoday.com/articles/319902.php, accessed on 10 May 2018). Obesity results due to imbalance between the energy content of food eaten and energy expended by the body to maintain life and to perform physical work. Such an energy balance framework is a potentially powerful tool for investigating the regulation of body weight.

Conversion of white adipose tissue to brown or beige/brite is reported as an effective mechanism to utilize the undue energy abundance and increasing the energy expenditure. The role of brown adipose tissue (BAT) is well described in the following prior arts:
1. Elattar. S and Satyanarayana, “Can Brown Fat Win the Battle against White Fat?”, J Cell Physiol. 2015 October; 230910):2311-7
2. Zafrir B, “Brown adipose tissue: research milestones of a potential player in human energy balance and obesity”, Horm Metab Res. 2013 October; 45(11):774-85).
3. Giralt M, Villarrova F “White, brown, beige/brite: different adipose cells for different functions?” Endocrinology. 2013 September; 154(9):2992-3000

Drugs and/or natural molecules that facilitate the conversion of white to brown adipocytes are effective in the treatment/management of obesity related conditions. However, we need a better understanding of the components involved in energy expenditure and their interactions over various time scales to explain the natural history of conditions such as obesity and to estimate the magnitude and potential success of therapeutic interventions. (Kevin D. Hall, Steven B. Heymsfield, Joseph W. Kemnitz, Samuel Klein, Dale A. Schoeller, and John R. Speakman, Energy balance and its components: implications for body weight regulation, Am J Clin Nutr. 2012 April; 95(4): 989-994).

Recently, it was observed that the gut microbiota is altered in conditions like obesity and type II diabetes. Administration of probiotics to obese individuals resulted in an effective weight loss. One particular gut microbe, Akkermansia muciniphila was inversely associated with obesity, diabetes, cardio metabolic diseases and low-grade inflammation (Cani et al., Next-Generation Beneficial Microbes: The Case of Akkermansia muciniphila, Front. Microbiol., 22 Sep. 2017, https://doi.org/10.3389/fmicb.2017.01765). Evidence show that the relative abundance of A. muciniphila increased more than 100-fold following the ingestion of prebiotics (Everard et al., 2014 Microbiome of prebiotic-treated mice reveals novel targets involved in host response during obesity. ISME J. 8, 2116-2130. doi: 10.1038/ismej2014.45). Studies also indicated that the number of A. muciniphila was found to be lower in obese and type 2 diabetic mice and increased with antidiabetic treatments (Cani et al., Next-Generation Beneficial Microbes: The Case of Akkermansia muciniphila, Front. Microbiol., 22 Sep. 2017, https://doi.org 10.3389/finicb.2017.01765). Another study observed that A. muciniphila treatment reversed high-fat diet-induced metabolic disorders, including fat-mass gain, metabolic endotoxemia, adipose tissue inflammation, and insulin resistance. (Amandine Everard, Clara Belzer, Lucie Geurts, Janneke P. Ouwerkerk, Celine Druart, Laure B. Bindels, Yves Guiot, Muriel Derrien, Giulio G. Muccioli, Nathalie M. Delzenne, Willem M. de Vos and Patrice D. Cani, Cross-talk between Akkermansia muciniphila and intestinal epithelium controls diet-induced obesity, PNAS May 28, 2013. 110 (22) 9066-9071). Hence, increasing the viable counts of Akkermansia muciniphila can be an effective therapy for the management of obesity, diabetes and other metabolic disorders. The probiotic and beneficial effects of Akkermansia muciniphila are well described in the following prior art documents.
1. Cani et al., Next-Generation Beneficial Microbes: The Case of Akkermansia muciniphila, Front. Microbiol., 22 Sep. 2017, https://doi.org/10.3389/fmicb.2017.01765
2. Gómez-Gallego et al., Akkermansia muciniphila: a novel fimunctional microbe with probiotic properties, Beneficial Microbes, 2016; 7(4): 571-584

Natural molecules are currently evaluated extensively for the management of obesity and related disorders. Extracts of Garcinia cambogia, have been reported to have a weight loss potential. U.S. Pat. No. 7,063,861, discloses a weight loss composition containing garcinol and hydroxycitric acid (HCA) and optionally with anthocyanins. U.S. Pat. No. 8,329,743 also discloses a weight loss formulation containing garcinol, pterostilbene and anthocyanin. U.S. Pat. No. 7,063,861 indicates that garcinol and HCA combination increases the bioavailability of HCA bringing about an anti-obesity effect. Hence, the anti-obesity effect of garcinol per se is not reported and also cannot be anticipated from the prior art documents. Moreover, Heo et al., (Gut microbiota Modulated by Probiotics and Garcinia cambogia Extract Correlate with Weight Gain and Adipocyte Sizes in High Fat-Fed Mice Sci Rep. 2016; 6:33566), reports the modulation of gut microbiota and increase in A. muciniphila by Garcinia cambogia extract without specific reference to garcinol. The present invention solves the above problem by disclosing the anti-obesity effect and the ability of modulating the gut microbiome by garcinol.

The principle objective of the invention is to disclose the anti-obesity effect of garcinol by bringing about weight loss and energy balance.

It is another objective of invention to disclose the ability of garcinol to modify the gut microbiome and increasing the viable counts of probiotic bacteria Akkermansia muciniphila.

It is yet another objective of invention to disclose the hypolipidemic effects of garcinol.

The present invention fulfils the above such objectives and provides further related advantages.

### SUMMARY OF THE INVENTION

The present invention pertains to garcinol compositions for obesity management. More specifically, the invention relates to the use of garcinol for a) the maintaining energy balance in mammalian adipose cellular systems b) management of hypercholesterolemia and c) reducing weight gain in mammals. The modification of gut microbiota and the increase of beneficial microbe, Akkermansia muciniphila by garcinol is also disclosed.

### BRIEF DESCRIPTION OF THE DRAWINGS

FIG. 1a is the oil-O-red staining of adipocytes indicating a dose dependent reduction in lipid accumulation in adipocytes by garcinol.

FIG. 1b is the graphical representation of the percentage inhibition of adipogenesis by garcinol.

FIG. 2 is a graphical representation showing the decrease in expression of genes related to adipogenesis in garcinol treated groups.

FIG. 3 is a graphical representation showing the increase in expression of genes related to brown fat conversion and fat utilization in garcinol treated groups.

FIG. 4a is a graphical representation showing the change in weight of animals administered with different concentrations of garcinol over a period of 4 months.

FIG. 4b is graphical representation showing the final weight of animals administered with different concentrations of garcinol for a period of 120 days.

FIG. 5 is a representative image showing the different fat pad regions in the mice body.

FIG. 6 represents the change in the weight of peritoneal, mesenteric and perigonadal fat tissues treated with different concentrations of garcinol.

FIG. 7 is a graphical representation showing the percentage reduction in visceral fat in animals administered with different concentrations of garcinol in a dose dependent manner.

FIGS. 8a and 8b are graphical representations showing the decrease in expression of genes related to adipogenesis in adipose tissues of animals administered with different concentrations of garcinol.

FIG. 9 is a graphical representation showing the increase in expression of genes related to brown fat conversion and fat utilization in adipose tissues of animals administered with different concentrations of garcinol.

FIG. 10a is a graphical representation showing the levels of total cholesterol and triglycerides in serum of animals administered with different concentrations of garcinol.

FIG. 10b is a graphical representation showing the levels of LDL and VLDL in serum of animals administered with different concentrations of garcinol.

FIG. 10c is a graphical representation showing the levels of HDL in serum of animals administered with different concentrations of garcinol.

FIG. 11 shows the experimental design for anti obesity study with Garcinol in HFD-induced Obesity Mice.

FIG. 12 is a representative image showing the effect of Garcinol on HFD-induced Obesity in C57BL/6 mice. Image A is the representative photographs of each group of mice at the end of week 13. Image B shows the Photographs of perigonadal adipose tissues and image C shows photographs of the liver.

FIG. 13 is a graphical representation of body weight of animal administered with various concentrations of garcinol. Body weight was monitored weekly and the average body weight of each group was expressed as the means±SE, p<0.05; a, b, c, and d significantly differed between each group.

FIG. 14a shows the photographs of perigonadal, retroperitoneal, and mesenteric adipose tissues of animals administered with garcinol.

FIG. 14b is the graphical representation of adipose tissue weights of animals administered with garcinol.

FIG. 15a shows the representative images of each study group for the pathological assessment by H&E staining in perigonadal adipose tissue.

FIG. 15b is graphical representation showing the percentage frequency of adipocyte size on animals treated with garcinol. Adipocyte size was quantified under the microscope from representative sections.

FIGS. 16a and 16b show the change in the taxonomic composition of colonic bacterial communities in animals administered with garcinol. FIG. 16a shows the change in the phylum and FIG. 16b represents the genus relative abundance of fecal microbiota.

FIGS. 17a and 17b represents Principal Coordinate Analysis (PCoA) plots showing the normalized relative abundance of all samples (A) Phylum. (B) Genus

FIG. 17c represents the Heatmap showing the abundance of 50 operational taxonomic units (OTUs) significantly altered by garcinol in HFD-fed mice.

FIG. 18a shows the effects of garcinol on protein expression of adipocyte specific factors and AMPK signaling in HFD-fed C57BL/6 Mice Perigonadal Adipose Tissue. The protein levels of p-AMPK (Thr172), AMPK, Pref-1, SREBP-1 and PPARγ were determined by Western blot analysis with specific antibodies. β-actin was used as a loading control.

FIG. 18b is the graphical representation of the level of protein expression of adipocyte specific factors and AMPK signaling in HFD-fed C57BL/6 Mice Perigonadal Adipose Tissue. The values indicate the relative density of the protein band normalized to β-actin.*p<0.05;**p<0.005; compared with the HFD treatment only.

FIG. 19a is a graphical representation of body weight of animal administered with various concentrations of garcinol and garcinol blend.

FIG. 19b is the representative photographs of each group of mice at the end of the study period.

FIG. 20a is a graphical representation of perigonadal fat weights of animals administered with different concentrations of garcinol and garcinol blend.

FIG. 20b is a graphical representation of retroperitoneal fat weights of animals administered with different concentrations of garcinol and garcinol blend.

FIG. 20c is a graphical representation of mesentric fat weights of animals administered with different concentrations of garcinol and garcinol blend.

### DESCRIPTION OF THE MOST PREFERRED EMBODIMENTS

In the most preferred embodiment, the present invention discloses a method for therapeutic management of obesity in mammals, said method comprising steps of administering effective concentration of a composition containing garcinol to said mammals to bring about a) inhibition of adipogenesis b) decrease in body weight and visceral fat in said mammals. In a related embodiment, inhibition of adipogenesis in brought about by down regulation of genes selected from the group consisting of, but not limited to, peroxisome proliferator-activated receptor gamma (PPARγ), CCAAT/enhancer binding protein alpha (cEBPα), first apoptotic signal (FAS), adipocyte protein 2 (AP2), resistin and leptin. In a related embodiment, inhibition of adipogenesis is brought about by up regulation of genes selected from the group consisting of, but not limited to, phospho-adenosine monophosphate-activated protein kinase (p-AMPK), AMP-activated protein kinase (AMPK) and Preadipocyte factor 1 (PREF-1). In another related embodiment, the visceral fat is selected from the group consisting of mesenteric fat, peritoneal fat and perigonadal fat. In a related embodiment, the composition is formulated with pharmaceutically/nutraceutically acceptable excipients, adjuvants, diluents or carriers and administered orally in the form of tablets, capsules, syrups, gummies, powders, suspensions, emulsions, chewable, candies and eatables.

In another preferred embodiment, the invention discloses a method of achieving energy balance in mammalian adipose cellular systems, said method comprising step of administering composition containing garcinol in effective amounts targeted towards mammalian pre-adipocytes and/or adipocytes to achieve effects of (a) increased inhibition of adipogenesis and (b) increased expression of factors that function individually or in combination to specifically recruit brown adipocytes or brown like (beige or brite) adipocytes, c) induce brown like phenotype (beige or brite adipocytes) in white adipocyte depots, to bring about the effect fat utilization and energy balance in said mammals. In related embodiments, the factors include the transmembrane protein mitochondrial uncoupling protein (UCP-1), the transcription co-regulators PR domain containing protein 16 (PRDM16) and Peroxisome proliferator-activated receptor gamma coactivator i-alpha (PGC-1α) which regulate the genes involved in energy metabolism and bone morphogenic protein 7 (BMP7), secretory protein controlling energy expenditure. In a related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the present invention discloses a method of modifying the gut microbiota in mammals, said method comprising step of administering effective amounts of a composition containing garcinol to said mammals to bring about change in the gut microbiota. In a related embodiment, the gut microbiota is selected from the Phylum Deferribacteres, Proteobacteria, Bacteroidetes, Verrucomicrobia and Firmicutes. In another related embodiment, the gut microbiota is selected from the genus Lactobacillus, Butyrivibrio, Clostridium, Anaerobranca, Dysgonomonas, Johnsonella, Ruminococcus, Bacteroides, Oscillospira, Parabacterroides, Akkermanisa, and Blautia. More specifically, the gut microbiota is selected from the group consisting of Parabacteroides goldsteinii, Bacteroides caccae, Johnsonella ignava, Blautia wexlerae, Dysgonomonas wimpennyi, Blautia hansenni, Anaerobranca zavarinni, Oscilospira eae, Mucispirillus schaedleri, Blautia coccoides, Anaeroiruncus colihominis, Butyrivibro proteoclasticus, Akkermansia muciniphila, Lachnospora pectinoschiza, Pedobacter kwangyangensis, Alkaliphilus crolonatoxidans, lactobacillus salivarius, Anaerivibria lipolyticus, Rhodothermus clarus, Bacteroides stercorirosoris, Ruminocococcus flavefaciens, Bacteroides xylanisolvens, Ruminococcus gnavus, Clostridium termitidis, Clostridium alkalicellulosi, Emticicia oligoraphica, Pseudobutyrivibm xylanivorans, Actinomyces naturae, Peptoniphilus coxii, and Dolichospermum curvum. In a related embodiment, modification of gut microbiota is effective in therapeutic management of diseases selected from the group consisting of obesity, cardiovascular complications, inflammatory bowel disease, Crohn's disease, Celiac disease, metabolic syndrome, liver diseases and neurological disorders. In a related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the invention discloses a method for increasing the viable counts of Akkermansia muciniphila in the gut of mammals, said method comprising steps of administering effective amounts of a composition containing garcinol to mammals to bring about an increase in the colonies of said bacteria. In a related embodiment, the increase in the colony counts of Akkermansia muciniphila reduces body weight through the AMPK signaling pathway by causing endocannabinoid release. In a related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the invention discloses a method of therapeutic management of hyperlipidemia in mammals, said method comprising step of administering an effective concentration of a composition containing garcinol to bring about the effects of (i) reducing the amount of total blood cholesterol levels; (ii) reducing the concentrations of low density lipoproteins (LDL) and very low density lipoproteins (VLDL); (iii) increasing the concentrations of high density lipoproteins (HDL) and (iv) reducing concentrations of serum triglycerides, in the blood of said mammals. In a related embodiment, the medical cause of hyperlipidemia is obesity. In a related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the invention discloses a composition containing garcinol for use as a prebiotic agent.

Specific illustrative examples enunciating the most preferred embodiments are included herein below

### Example 1: Anti-Obesity Effects of Garcinol—Study Done at Sami Labs Limited, Bangalore, India and Srimad Andavan Arts & Science College, Tiruchirapalli, India

Adipogenesis Inhibition and Brown Fat Specific Gene Expression by Garcinol in Cultured 3T3L1 and Animal Tissues

Methodology

Preparation of Stock Solutions

Garcinol (20%) stock of 10 mg/ml was prepared in DMSO and filtered through 0.2 micron syringe filter. Stock was diluted 1000 times in DMEM to get 10 μg/ml final concentration and serially diluted. Insulin (Hi Media) was bought as a solution at a concentration of 20 mg/ml. This was diluted to 1 μg/ml in DMEM. IBMX—(Sigma)—Stock of 5 mM was prepared in DMEM, and diluted 10 times to be used at a final concentration of 0.5 mM. Dexamethasone (Sigma)—A stock of 10 μM was prepared in DMEM and diluted 40 times to get a final concentration of 0.25 μM

Cell Culture

Mouse 3T3-L1 pre-adipocytes were cultured in DMEM containing 25 mM glucose with 10% heat-inactivated fetal calf serum with antibiotics at 37° C. and 5% CO2. When the cells were 70-80% confluent, they were trypsinized, washed and seeded in 6 well plates at a density of 2×10⁶ cells per well. Cells were induced to differentiate 2 d after reaching confluence (day 0), by supplementing DMEM media containing 10% Fetal Bovine Serum(FBS) along with 1 μg/mL insulin, 0.25 μM dexamethasone, 0.5 mM 1-methyl-3-isobutyl-xanthine (IBMX) and different concentrations of Garcinol (20%). From day 3 until day 7, cells were maintained in progression media supplemented with 1 μg/mL insulin and different concentrations of Garcinol (20%). Untreated cells and undifferentiated cells grown in FCS media were taken as Adipogenesis positive and negative controls for the experiment. Quantification for amount of triglycerides accumulated in adipocytes was done by Oil red O staining.

RNA Extraction

Cells were harvested after second progression on day 7 and total RNA was extracted using the Trizol method. Extracted RNA was treated with DNAse I to remove any contaminating DNA and again extracted using phenol:chloroform:isoamyl alcohol extraction (24:25:1). Quality of RNA was determined by checking the absorbance at 260/280 nm using a Nanodrop (Thermo)

Gene Expression Studies in Mouse Fat Pad

The frozen fat pads from treated and untreated animals were collected in RNA later and frozen. Approximately 100 mg of the tissue was homogenized in ice and extracted with 1 ml Trizol as described earlier.

Quantitative Real Time PCR

2 μg of total RNA was taken for cDNA synthesis using SuperScript III First-Strand Synthesis System (Life Technologies). Quantitative RT-PCR analysis was performed to determine the expression of brown fat specific genes in Roche Light cycler 96 using SYBR Green master mix (Thermo Scientific). β actin was used as a house keeping gene The relative RNA abundance of BAT genes was normalized to the housekeeping β actin gene and expressed as delta delta CT (equivalent to fold change transformed by Log 2).

Primer sequence: The primers used for the determining the expression of brown fat specific genes and genes related to adipogenesis is given in table 1

TABLE 1
Primers used for analyzing the expression
of BAT and adipogenesis specific genes

| | |
|---|---|
| Name | Primer sequence |

BAT specific Genes

| | |
|---|---|
| m Ucp1 F | AGGCTTCCAGTACCATTAGGT |
| m Ucp1 R | CTCAGTGAGGCAAAGCTGATTT |
| mpgc1αF | CCC TGC CAT TGT TAA GAC C |
| mpgc1αR | TGC TGC TGT TCC TGT TTT C |
| mprdm16 F | TCCCACCAGACTTCGAGCTA |
| mprdm16 R | ATCTCCCATCCAAAGTCGGC |
| mBMP7 F | GAGGGCTGGTTGGTGTTTGAT |
| mBMP7 R | GTTGCTTGTTCTGGGGTCCAT |
| m βactin F | GAAGTCCCTCACCCTCCCAA |
| m βactin R | GGCATGGACGCGACCA |

Adipogenesis

| | |
|---|---|
| m PPAR g F | TCGCTGATGCACTGCCTATG |
| m PPAR g R | GAGAGGTCCACAGAGCTGATT |
| m c/EBP a F | CAAGAACAGCAACGAGTACCG |
| m c/EBP a R | GTCACTGGTCAACTCCAGCAC |
| m FAS F | CTGAGATCCCAGCACTTCTTGA |
| m FAS R | GCCTCCGAAGCCAAATGAG |
| m AP2 F | CATGGCCAAGCCCAACAT |
| m AP2 R | CGCCCAGTTTGAAGGAAATC |

Results

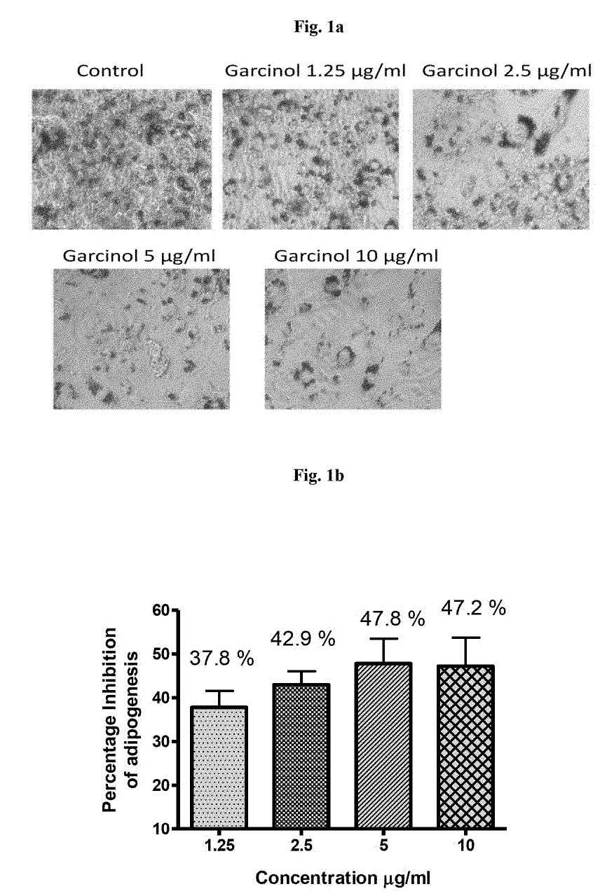

The lipids accumulated in adipocytes were quantified by Oil red O staining. Garcinol showed a dose dependent reduction in lipid accumulation in adipocytes (FIG. 1) with 5 and 10 μg/ml showing the highest inhibition of lipid accumulation by 47.8% and 47.2% (FIG. 1b).

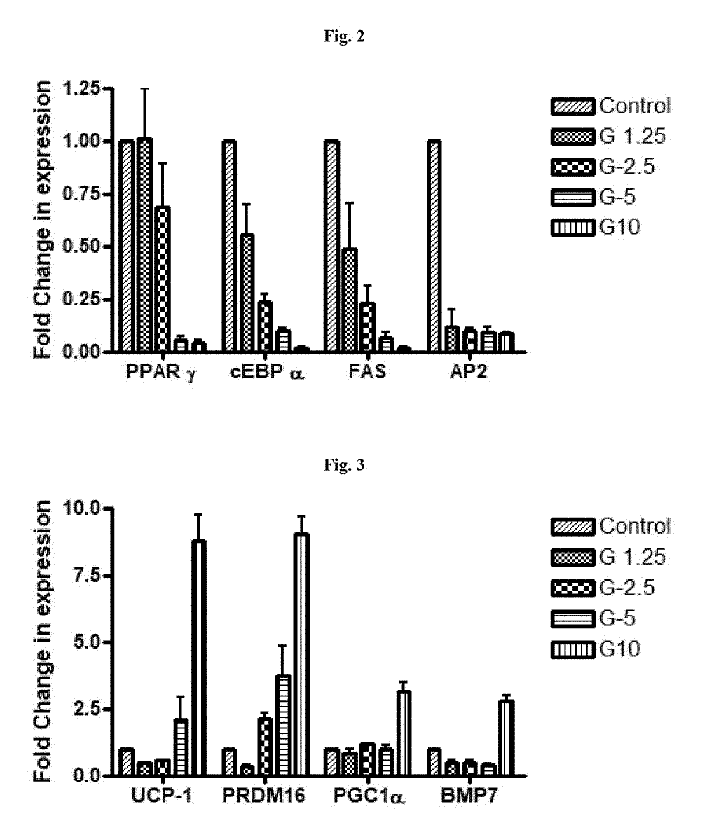

With respect to the genes involved in adipogenesis, PPARγ is considered to be the master regulator of adipogenesis. Decrease in PPARγ Expression will reduce the expression of other adipogenesis specific genes. In the present study, garcinol exhibited a dose expended reduction in the PPARγ Expression and the expression genes related to adipogenesis and fatty acid synthesis like cEBPα, FAS and AP2 (FIG. 2), indicating that garcinol inhibits adipogenesis in a dose dependent manner.

Garcinol also significantly increased the brown adipose tissue specific genes. The expression of UCP1, PRDM16, PGC1α and BMP7 was increased by garcinol in a dose dependent manner (FIG. 3) suggesting that garcinol is effective in converting the white adipose tissue depots to brown or brite/beige adipose tissue thereby increasing energy expenditure by fat utilisation and lipolysis.

Effect of Garcinol on High Fat Diet Induced Obesity in C57 Mice

Methods

Animals—

C57/BL6 mice, 6-8 weeks of age and 8 animals/Group (4 Male and 4 Female) were used for the study. Animals were housed under standard laboratory conditions, air-conditioned with adequate fresh air supply (12-15 Air changes per hour), room temperature 20.2-23.5° C. and relative humidity 58-64% with 12 hours fluorescent light and 12 hours dark cycle. The temperature and relative humidity was recorded once daily.

Feed

The animals were fed with Normal diet (9 kcal/day) and High fat diet (50 kcal/day) throughout the acclimatization and experimental period.

Water was provided along with High Fat Diet to the animals throughout the acclimatization and experimental period. Water from water filter curn purifier was provided in animal feeding bottle with stainless steel sipper tubes.

All the studies were conducted according to the ethical guidelines of CPCSEA after obtaining necessary clearance from the committee (Approval No: 790/03/ac/CPCSEA).

a. In accordance with the recommendation of the Committee for the Purpose of Control and Supervision of Experiments on Animals (CPCSEA) guidelines for laboratory animal facility published in the gazette of India, Dec. 15, 1998.
b. The CPCSEA approval number for the present study (Anti-obesity activity) is SAC/IAEC/BC/2017/IP.-001.

The design of the study groups is depicted in table 2.

TABLE 2
Study design (16 weeks)

| Groups | Treatment | | Diet | |
|---|---|---|---|---|
| G1 | None | | Normal diet | (9 kcal % fat) |
| G2 | None | | high fat diet | (50 kcal % Fat) |
| G3 | Garcinol | (5 mg/kgbw) | high fat diet | (50 kcal % Fat) |
| G4 | Garcinol | (10 mg/kgbw | high fat diet | (50 kcal % Fat) |
| | 16 weeks. | | | |
| G5 | Garcinol | (20 mg/kgbw) | high fat diet | (50 kcal % Fat) |
| | for 16 weeks. | | | |
| G6 | Garcinol | (40 mg/kgbw) | high fat diet | (50 kcal % Fat) |

Body weight of the animals was recorded in all the days of experimental period. At the end of the experimental period, the animals were sacrificed by cervical dislocation. Blood was collected and Serum was separated by centrifugation and used for the analysis of biochemical parameters. The organs such as Liver Kidney, Spleen and Pancreas and Fat Tissues (Retroperitoneal, Peri-gonadal and Mesenteric) were dissected out and washed in phosphate buffered saline.

Efficacy Measurement

The following parameters were measured in the above groups:
Measurement of Body weight
Determination of Organ Weight
Estimation of Cholesterol (Zak et al., (2009) A new method for the determination of serum cholesterol. J Clin Endocrinol Metab., 94(7), 2215-2220)
Estimation of Triglycerides (Foster L. B and Dunn R. T. (1973) Stable reagents for determination of serum triglycerides by a colorimetric Hantzsch condensation method. Clin Chem, 196, 338-340).
i) Estimation of HDL Cholesterol (Burstein et al., (1970). Determination of HDL cholesterol. J. lipid Res., 11, 583).
Determination of LDL and VLDL (Friedewald et al., (1972) Estimation of the concentration of Low Density Lipoprotein cholesterol in plasma without use of preparative centrifuge. J. Clin Chem.; 18:499).

Results

Body Weight

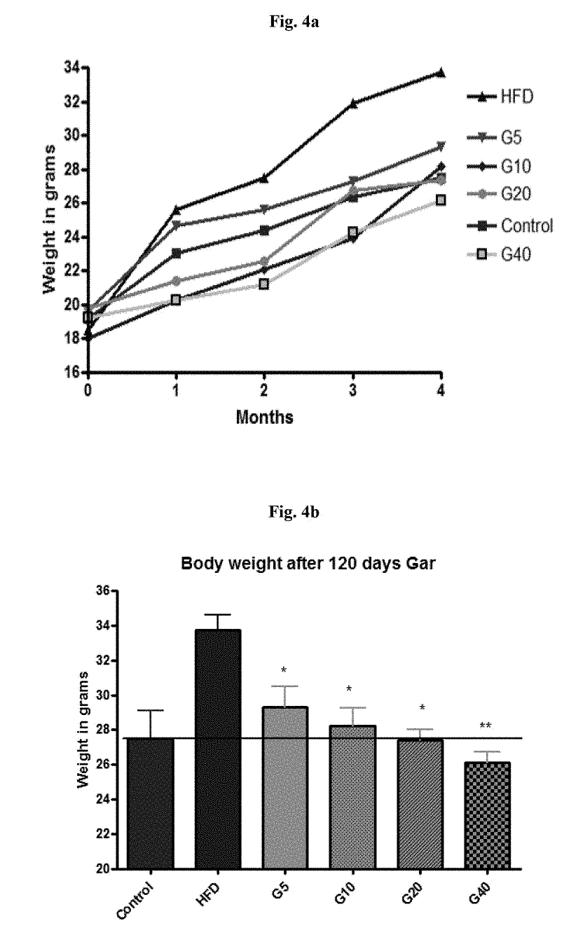

The results indicated that garcinol inhibited weight gain in a dose dependant manner in the animals fed with high fat diet (FIGS. 4a and 4b) for a period of 120 days. The percentage change in weight is depicted in the below table.

TABLE 3
Change in weight of the study animals

| | Control | HFD | G5 | G10 | G20 | G40 |
|---|---|---|---|---|---|---|
| Initial | 19.13 ± 0.91 | 18.50 ± 0.92 | 19.63 ± 0.94 | 18.00 ± 0.92 | 19.75 ± 0.88 | 19.25 ± 0.79 |
| weight (g) | | | | | | |
| Final | 27.5 ± 1.37 | 33.75 ± 1.60 | 29.33 ± 1.47 | 28.25 ± 1.39 | 27.37 ± 1.88 | 26.00 ± 1.33 |
| Weight (g) | | | | | | |
| Change in | 8.37 ± 1.41 | 15.25 ± 1.92 | 10.03 ± 2.25 | 10.25 ± 1.39 | 7.62 ± 2.18 | 6.28 ± 1.03 |
| weight (g) | | | | | | |

Reduction in Fat Deposits

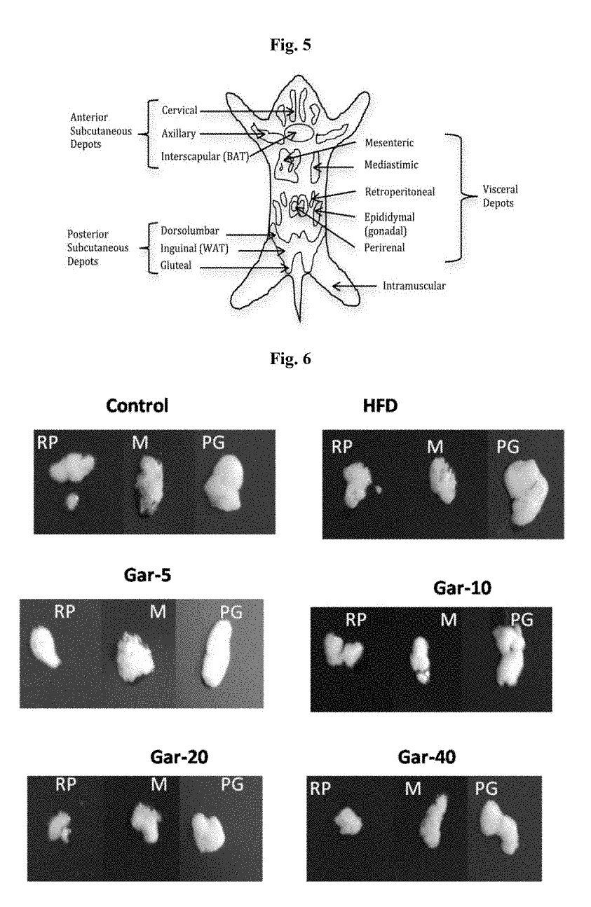

The fat reduction in the different fat pad regions of mice (FIG. 5) was also evaluated. The weights of Retroperitoneal, Peri-gonadal and Mesenteric Fat deposits after the 120 day administration of garcinol is tabulated as below

TABLE 4
Effect of Garcinol on Fat weight of HFD induced Mice

| | Retroperitoneal | Peri-gonadal Fat | Mesenteric Fat |
|---|---|---|---|
| Groups | Fat (g wet tissue) | (g wet tissue) | (g wet tissue) |
| I | 0.41 ± 0.03 | 1.14 ± 0.19 | 0.55 ± 0.03 |
| II | 0.55 ± 0.06 | 2.01 ± 0.22 | 0.69 ± 0.08 |
| III | 0.45 ± 0.06 | 1.33 ± 0.32 | 0.63 ± 0.07 |
| IV | 0.43 ± 0.05 | 1.17 ± 0.22 | 0.56 ± 0.05 |
| V | 0.46 ± 0.06 | 1.35 ± 0.21 | 0.601 ± 0.08 |
| VI | 0.46 ± 0.04 | 1.41 ± 0.14 | 0.59 ± 0.05 |

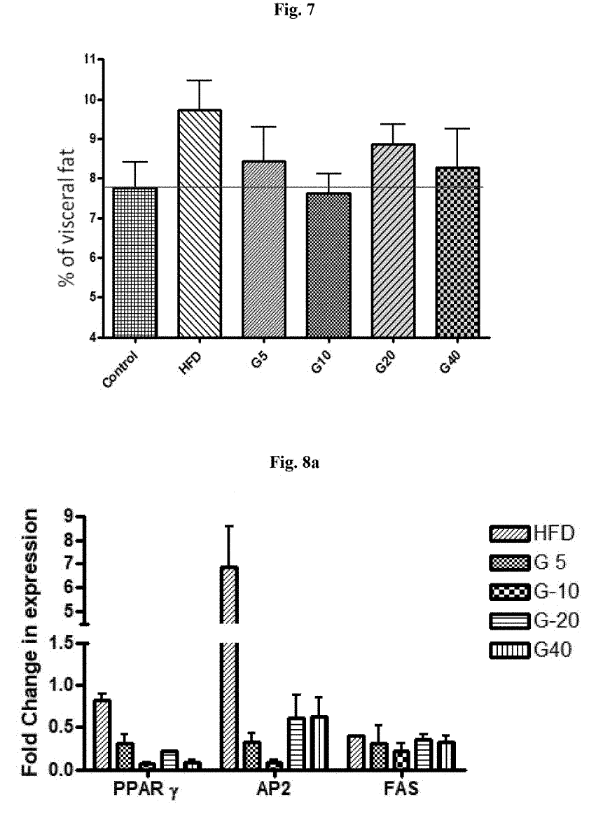

Garcinol treatment significantly reduced fat accumulation in the different fat pad regions (FIG. 6). Percentage of Visceral fat was reduced by garcinol treatment (FIG. 7) with the dose of 10 mg/kg body weight showing the maximum effect.

Organ Weights

Garcinol administration did not adversely affect the weight of the organs, suggesting that garcinol does not induce any adverse effects in critical organs. (Table 5).

TABLE 5
Weights of kidney, spleen and pancreas in garcinol treated animals

| | Kidney weight | Spleen Weight | Pancreas Weight |
|---|---|---|---|
| Groups | (g wet tissue) | (g wet tissue) | (g wet tissue) |
| I | 0.42 ± 0.02 | 0.19 ± 0.01 | 0.15 ± 0.01 |
| II | 0.553 ± 0.06 | 0.26 ± 0.03 | 0.24 ± 0.02 |
| III | 0.49 ± 0.03 | 0.25 ± 0.02 | 0.23 ± 0.01 |
| IV | 0.42 ± 0.03 | 0.20 ± 0.01 | 0.17 ± 0.01 |
| V | 0.45 ± 0.06 | 0.22 ± 0.03 | 0.21 ± 0.02 |
| VI | 0.42 ± 0.04 | 0.23 ± 0.02 | 0.22 ± 0.02 |

Gene Expression:

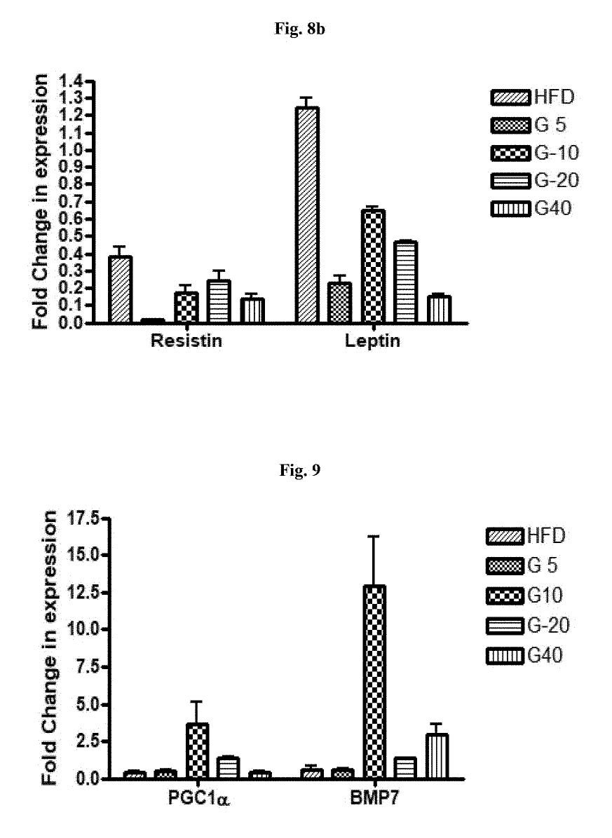

Reduction in the expression of genes related to adipogenesis was observed in fat pad of animals treated with Garcinol. Similar to Mouse 3T3-L1 cell lines, garcinol administration significantly reduced the expression of PPARγ, AP2, FAS, RESISTIN and LEPTIN in the fat pad regions (FIGS. 8a and 8b). Similarly, garcinol administration effectively increased the expression of Brown fat specific genes in the mice fat pad regions (FIG. 9).

Lipid Profile:

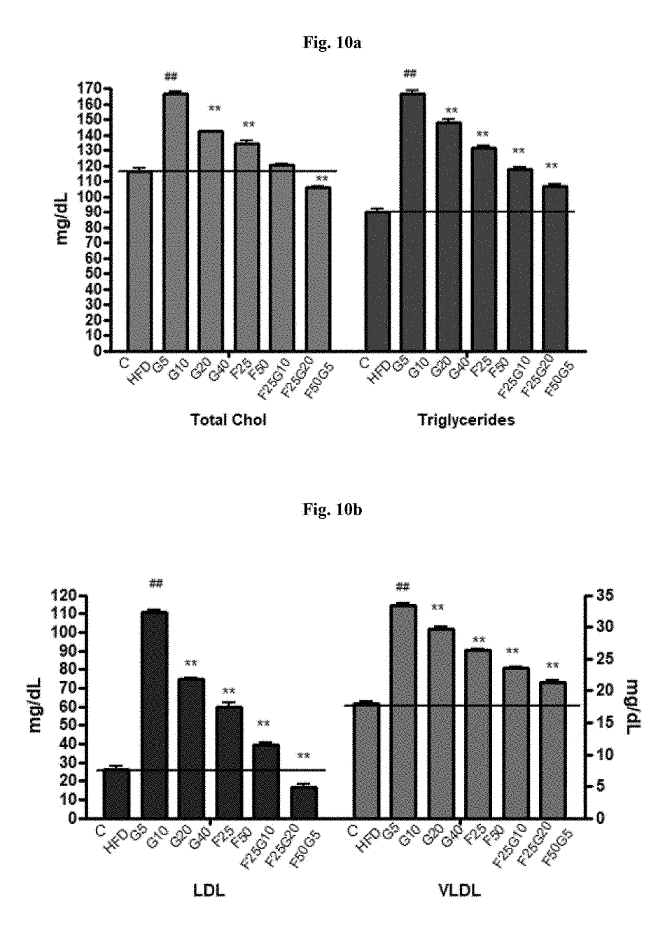

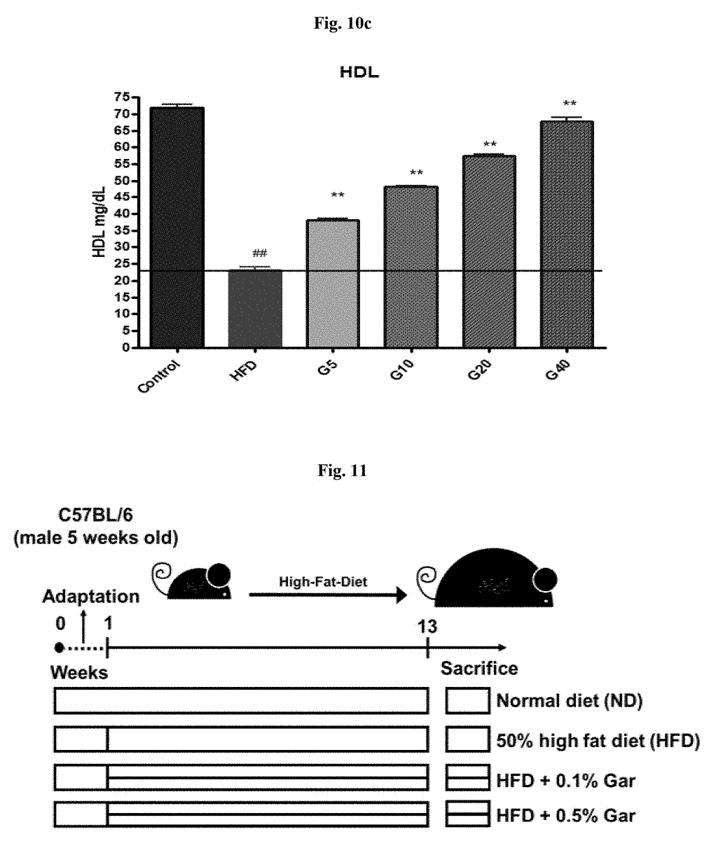

The high fat diet increased the levels of total cholesterol, LDL, VLDL and triglycerides in the serum of study animals. High fat diet, co administered with garcinol, significantly reduced the total cholesterol and triglycerides (FIG. 10a), LDL and VLDL (FIG. 10b) and increased the HDL levels (FIG. 10c) in the serum.

Conclusion:

Garcinol treatment showed a dose dependent inhibition of adipogenesis in vitro and induced the conversion of white adipose tissue to brown or brite/beige thereby increasing fat utilisation and energy metabolism. The in vivo results indicated that Garcinol was effective in significantly reducing body weight and visceral fat accumulation at 10 mg/kg and reduced adipogenesis specific gene expression and increased brown adipose tissue specific genes in fat pad in mouse fat pads. Garcinol administration also resulted in the reduction of visceral fat and organ weights indicating that garcinol promotes lipolysis and energy metabolism. Over all, garcinol induces weight loss, reduces visceral fat and maintains health of key organs.

### Example 2: Anti-Obesity Effects of Garcinol—Study Done at National Taiwan University, Taipei, Taiwan

Methodology

Reagents and Antibodies

AMPK and p-AMPK (Thr172) antibodies were purchased from Cell Signaling Technology (Beverly, Mass., USA). The SREBP-1 antibody was procured from Santa Cruz Biotechnology (Santa Cruz, Calif., USA). The PPARγ and Pref-1 antibodies were purchased from abcam (Cambridge, England). The mouse β-actin monoclonal antibody was obtained from Sigma Chemical Co (St. Louis, Mo., USA). The Bio-Rad protein assay dye reagent was purchased from Bio-Rad Laboratories (Munich, Germany). Xylene and hematoxylin and eosin (H&E) stain were acquired from Surgipath (Peterborough, UK). Cholesterol used as part of the animal diet was obtained from Acros Organics (Bridgewater, N.J., USA). Garcinol was procured from Sabinsa Corp. (East Windsor, N.J., USA). The purity of garcinol was determined by high-performance liquid chromatography (HPLC) to be higher than 99%.

Animal Care and Experimental Design

Five-week-old male C57BL16 mice were purchased from the BioLASCO Experimental Animal Center (Taiwan Co., Ltd, Taipei, Taiwan) and housed in a controlled atmosphere (25±1° C. at 50% relative humidity) with a 12-h light/dark cycle. After one week of acclimation, animals were randomly distributed into normal diet (ND, 15% energy as fat), HFD (50% energy as fat), and HFD with 0.1% or 0.5% garcinol groups of eight mice in each group for 13 weeks. The experimental design is summarized in FIG. 11. The experimental diets were modified from the Purina 5001 diet (LabDiet, PMI Nutrition International, St. Louis, Mo., USA). The animals had ad libitum access to food and water. Food cups were replenished with a fresh diet every day. All animal experimental protocols employed in this study were approved by the Institutional Animal Care and Use Committee of the National Taiwan University (IACUC, NTU). Upon termination of the study, the animals were sacrificed by CO, asphyxiation and dissected, and the weights of their whole bodies and selected tissues, including the liver, kidney, spleen, adipose tissues (perigonadal, retroperitoneal, and mesenteric fat) and serum were immediately collected, weighed, and photographed.

Histopathological Examination

A portion of perigonadal fat and the median lobe of the liver were dissected and fixed in 10% buffered formalin, dehydrated with a sequence of ethanol solutions, and processed for embedding in paraffin. Sections of 3-5 μm in thickness were cut, deparaffinized, rehydrated, stained with H&E, and subjected to photomicroscopic assessment. Adipocyte size was determined using Image J software (Rasband, W. S., ImageJ, U. S. National Institutes of Health, Bethesda, Md., USA).

Biochemical Analysis

Blood samples were collected from the left ventricle under anesthesia. The samples were mixed in 10 μL of heparin sodium and centrifuged at 3500 rpm and 4° C. for 10 min. The plasma was then stored at −80° C. until use. Glutamic-pyruvic transaminase (GPT), total cholesterol (TC), TG, high-density lipoprotein (HDL), and low-density lipoprotein (LDL) levels were analyzed at the National Laboratory Animal Center, NLAC (Taipei, Taiwan) on a 7080 Biochemical Analyzer (Hitachi, Tokyo, Japan) according to the manufacturer's instructions.

16S rDNA Gene Sequencing and Analysis

Total DNA was extracted from fresh fecal samples. The purified DNA was eluted using the innuSPEED Stool DNA kit (Analytik Jena AG, Jena, Germany) according to the manufacturer's protocol. The PCR primer sequences from Caporaso et al., (Caporaso, J. G., Lauber, C. L., Walters, W. A., Berg-Lyons, D. et al., Global patterns of 16S rRNA diversity at a depth of millions of sequences per sample. Proc. Natl. Acad., Sci U.SA 2011, 108 Suppl 1, 4516-4522) were used to amplify the 16S rRNA variable region, and the PCR conditions were performed as mentioned in Tung el al., (Tung, Y. C., Lin, Y. H., Chen, H. J., Chou, S. C. et al., Piceatannol Exerts Anti-Obesity Effects in C57BL/6 Mice through Modulating Adipogenic Proteins and Gut Microbiota. Molecules. 2016, 21) and Chou et al., (Chou, Y. C., Suh, J. H., Wang, Y., Pahwa, M. et al., Boswellia serrata resin extract alleviates azoxymethane (AOM)/dextran sodium sulfate (DSS)-induced colon tumorigenesis. Mol. Nutr Food Res. 2017, 61) Then, the amplicons were used to construct index-labeled libraries with the Illumina DNA Library Preparation kit (Illumina, San Diego, Calif., USA). The Illumina MiniSeq NGS System (Illumina) was employed to analyze more than 100,000 reads with paired-end sequencing (2×150 bp), and the metagenomics workflow classified organisms from the amplicon using a database of 16S rRNA data. The classification was based on the Greengenes database (https://greengenes.lbl.gov/). The output of the workflow was a classification of reads at several taxonomic levels: kingdom, phylum, class, order, family, genus, and species.

Protein Preparation and Western Blot

Tissues were homogenized in ice-cold lysis buffer (10% glycerol, 1% TritonX-100, 1 mM Na₃VO₄, 1 mM EGTA, 10 mM NaF, 1 mM Na₄P₂O₇, 20 mM Tris buffer (pH7.9), 100 μM β-glycerophosphate, 137 mM NaCl, and 5 mM EDTA) containing 1 Protease Inhibitor Cocktail Tablet (Roche, Indianapolis, Ind., USA) on ice for 1 h, followed by centrifugation at 17,500 g for 30 min at 4° C. The protein concentration was measured with the Bio-Rad protein assay (Bio-Rad Laboratories, Inc., Hercules, Calif., USA).

Statistical Analysis

Statistical evaluation of the significance of the differences between the groups of mice was assessed using the Student t-test. For experiments comparing multiple groups, the differences were analyzed by carrying out one-way analysis of variance (ANOVA) and Duncan's post-hoc test. Data are presented as the means+SE for the indicated number of independently performed experiments, and p values <0.05 were considered statistically significant. Principal component analysis (PCA) was conducted to visualize the differences between samples.

Results

Body Weight Gain

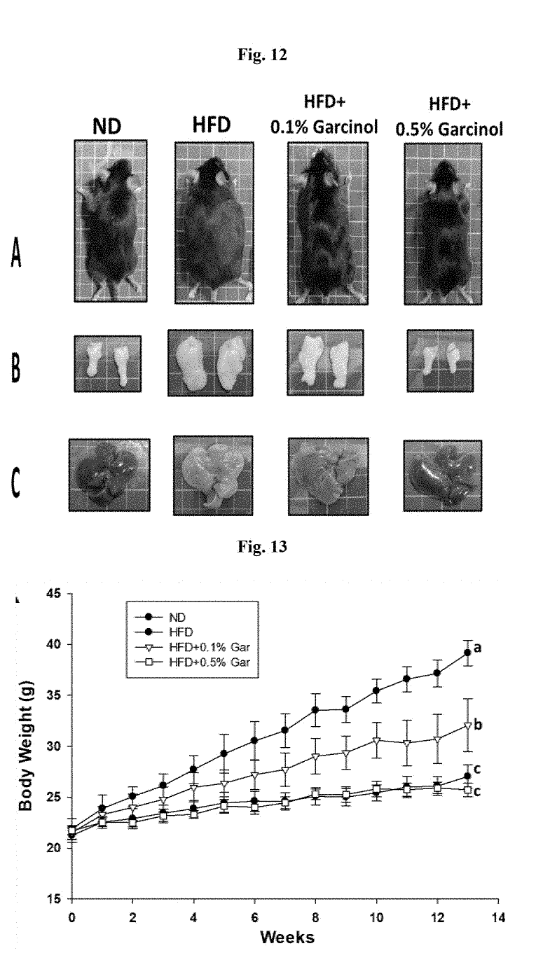

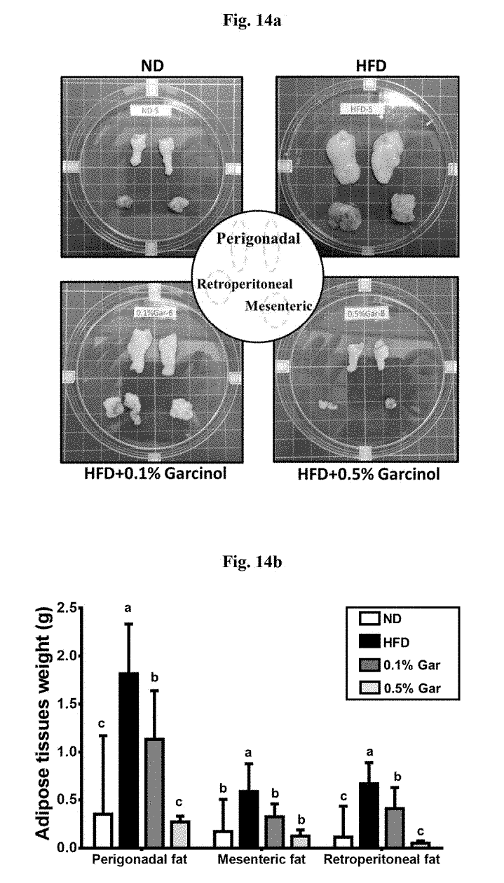

The results indicated that that HFD feeding for 13 weeks led to significant increase in body and liver weight along with perigonadal, retroperitoneal, and mesenteric fat accumulation. Diet supplemented with 0.1 and 0.5% garcinol reduced body weight in a dose-dependent manner. Co-treatment with high doses of garcinol (0.5%) and a HFD diet inhibited body weight gain, and there is no difference between HFD+0.5% garcinol and the ND group (FIG. 12 and FIG. 13).

Effect on White Adipose Tissue Adipocyte Size and Liver Homeostasis

Garcinol at concentrations of 0.5% dramatically decreased all three white adipose fat weights, compared to the HFD group, by 85.1% in terms of perigonadal weight, 92.4% retroperitoneal weight, and 77.7% mesenteric weight (FIGS. 14a and 14b).

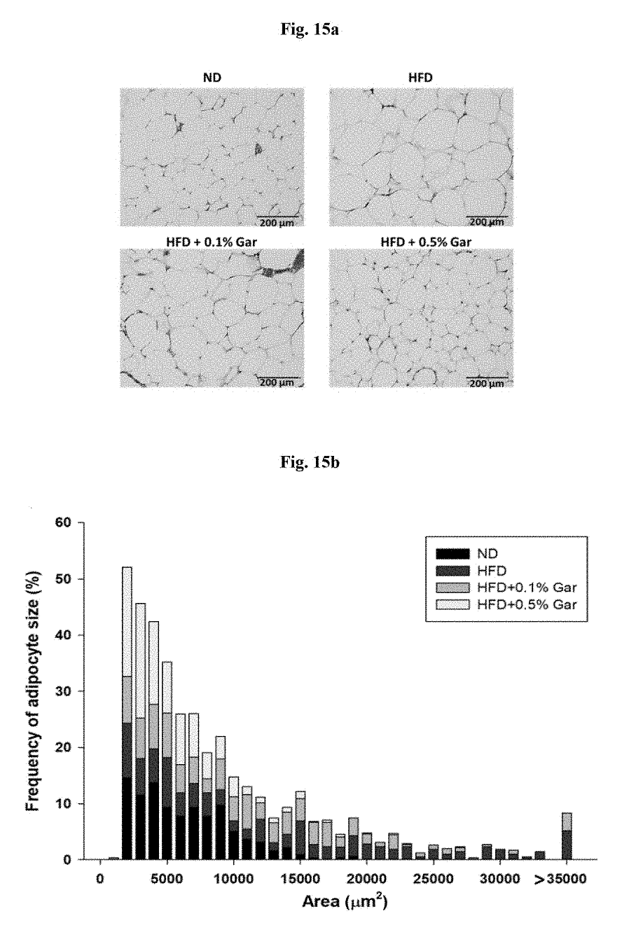

The average adipocyte size in perigonadal adipose tissue was evaluated by H&E staining, and the results revealed that adipocytes were enlarged in HFD-fed mice compared to those of ND mice. The increased adipocyte size was significantly reduced in the garcinol-treated mice (FIG. 15). Garcinol (0.5%) could prevent the enlargement of adipocytes induced by HFD, which made adipocyte distribution at a size of 2000 μm². Importantly, adipocyte size can be prevented or inhibited by garcinol in a dose-dependent fashion (Table 6).

TABLE 6
Effect of garcinol on adipocyte size

| Adipocye size | | | HFD + 0.1% | HFD + 0.5% |
|---|---|---|---|---|
| area (μm²) | ND | HFD | garcinol | garcinol |
| 2000 | 14.4 ± 2.6^{ab} | 9.84 ± 4.5^{b} | 8.38 ± 5.3^{b} | 19.2 ± 3.9^{a} |
| 15000 | 0.92 ± 0.4^{b} | 6.06 ± 2.5^{a} | 4.03 ± 1.0^{a} | 1.36 ± 0.9^{b} |
| >350000 | 0.00 ± 0.0^{c} | 5.10 ± 0.9^{a} | 3.32 ± 1.5^{b} | 0.00 ± 0.0^{c} |

The significance of the difference among the four groups was analyzed by one-way ANOVA and Duncan's multiple-range tests. The values with different letters are significantly different (p<0.05) between each group.

Lipid Profile:

The plasma lipid profiles were also analyzed and are presented in Table 7.

TABLE 7
Lipid profile in mice administered with garcinol

| | ND | HFD | HFD + 0.1% Gar | HFD + 0.5% Gar |
|---|---|---|---|---|
| GPT (U/L) | 27.2 ± 7.04^{ab} | 32.1 ± 6.42^{a} | 20.5 ± 7.26^{b} | 27.6 ± 3.72^{ab} |
| T-CHO (mg/dL) | 69.6 ± 7.31^{d} | 200.3 ± 11.30^{a} | 179.7 ± 11.85^{b} | 137.9 ± 11.78^{c} |
| TG (mg/dL) | 83.7 ± 14.56^{a} | 85.2 ± 13.09^{a} | 69.6 ± 19.90^{ab} | 55.6 ± 4.95^{b} |
| LDL (mg/dL) | 2.4 ± 0.41^{d} | 40.0 ± 2.89^{a} | 33.3 ± 0.72^{b} | 24.9 ± 4.47^{c} |
| HDL (mg/dL) | 57.8 ± 6.01^{c} | 155.6 ± 5.97^{a} | 152.6 ± 9.73^{a} | 118.7 ± 13.22^{b} |
| LDL/HDL | 0.04 ± 0.01^{c} | 0.25 ± 0.01^{a} | 0.22 ±+0.02^{b} | 0.21 ± 0.03^{b} |

Data are presented as the mean±SE. The significance of the differences among the four groups was analyzed by one-way ANOVA and Duncan's multiple-range tests. Values not sharing the same superscript letters in the same row are significantly different among groups. p<0.05; a, b, c, and d are significantly different between each group.

Mice administered with garcinol at 0.1% and 0.5% had significantly diminished serum levels of both TC and TG. With respect to LDL and HDL, garcinol (0.1 and 0.5%) could reduce LDL levels, induced by HFD, in a dose-dependent manner. As the TC decrease was brought about by HFD, the HFD group increases not only the LDL levels, but also HDL levels. Hence, we used the LDL/HDL ratio to express this change. High and low dosages of garcinol can significantly diminish the LDL/HDL ratio compared to the HFD group.

Garcinol Reversed HFD-Induced Gut Dysbiosis

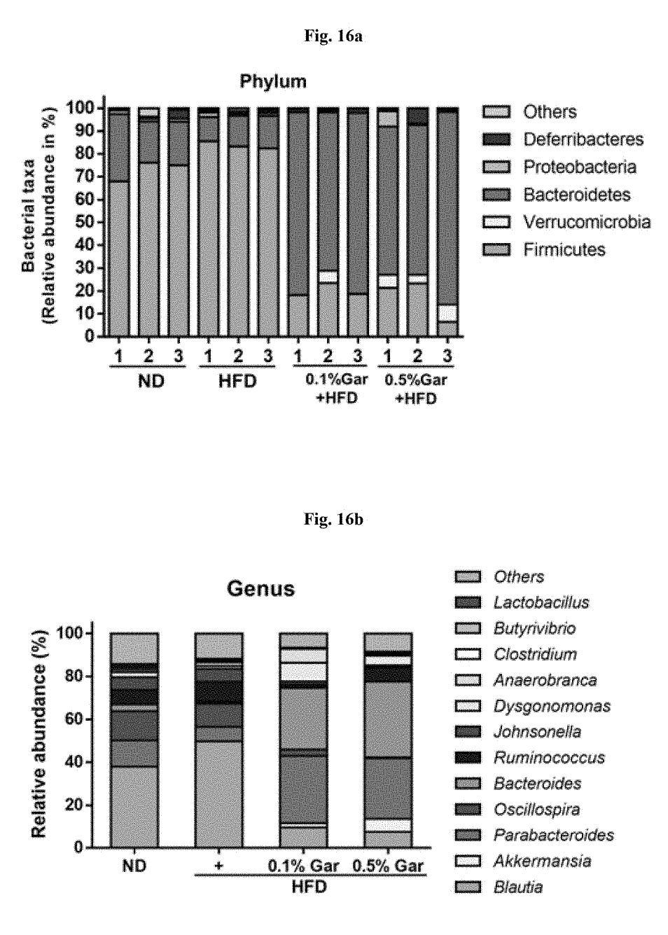

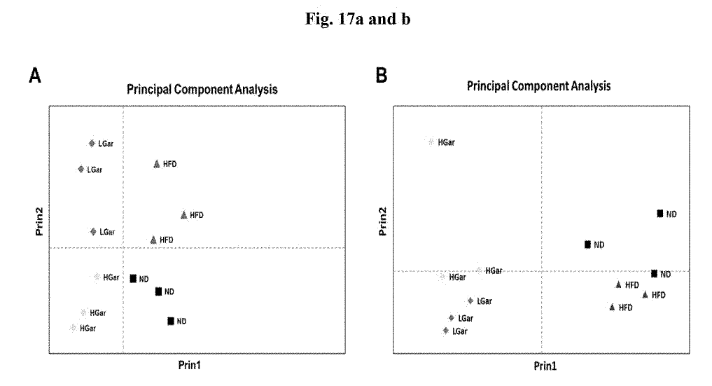

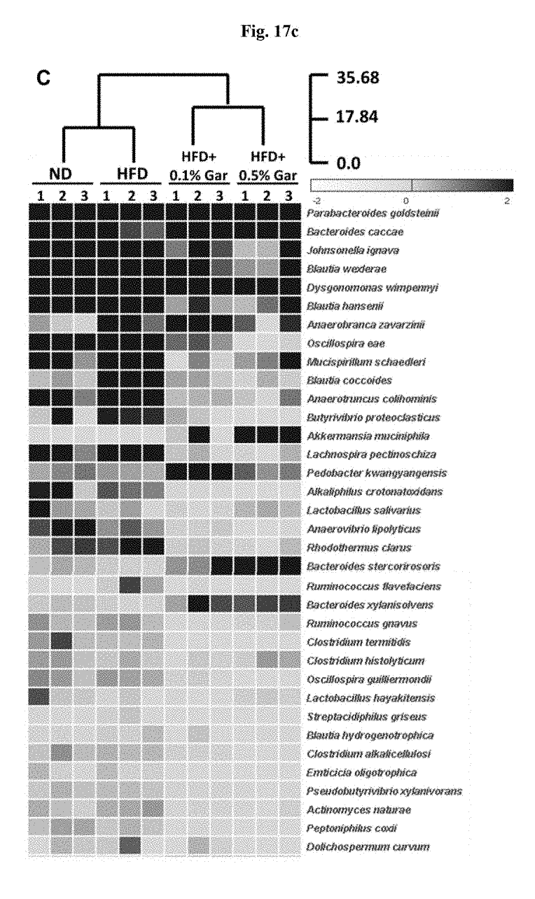

The overall composition of the bacterial community in the different groups was assessed by analyzing the degree of bacterial taxonomic similarity between metagenomic samples at the genus level. Bacterial communities were clustered using PCA, which distinguished microbial communities based on HFD diet/garcinol treatment. The gut microbiota of obese humans and HFD-fed mice is characterized by an increased Firmicutes-to-Bacteroidetes ratio (F/B ratio) (Brun, P., Castagliuolo, I., Di, L., V, Buda, A. et al., Increased intestinal permeability in obese mice: new evidence in the pathogenesis of nonalcoholic steatohepatitis. Am. J Physiol Gastrointest. Liver Physiol 2007, 292, G518-G525). The results indicated that the phylum level of HFD group has the higher F/B ratio compared with ND group (FIG. 16a). Interestingly, the Garcinol treatment reduced the F/B ratio by highly elevating Bacteroides communities. In addition, garcinol treatment made the Verrucomicrobia communities rise in number (FIG. 16b). PCA of UniFrac-based pair wise comparisons of community structures disclosed a distribution of the microbial community among the four groups of mice. The main finding of PCA was that different diets promoted the development of various gut microbial communities. HFD-fed mice formed a cluster that was distinct from ND group mice, and the HFD-fed mice were also distinct from garcinol treatment mice (FIGS. 17a, b and c). However, high doses of garcinol (0.5%) treated mice's microbial communities were closely clustered to that of ND mice, this indicates that garcinol has a marked effect on gut microbial community composition and also reversed HFD-induced gut dysbiosis.

Effects of Garcinol Administration on the Composition of Gut Microbial Communities

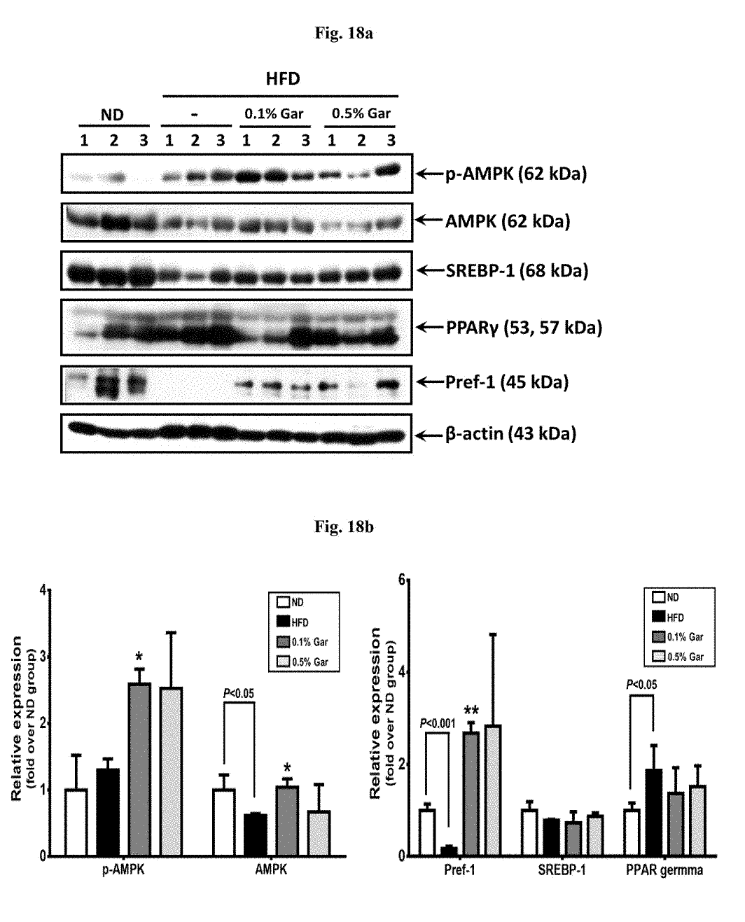

To further investigate whether the changes in the gut microbiota were induced by garcinol supplementation, we next determined the genus level of gut microbiota and used a heatmap to express the abundance of 50 OTUs significantly altered by garcinol in HFD-fed mice (FIG. 18). The results demonstrated that HFD-fed mice increased Blautia communities, which dramatically decreased in both high- and low-dose garcinol treatment groups. The studies pointed that Blautia spp. and Enterobacier spp. were related to a HFD causing obesity in a mouse model (Becker, N., Kunath, J., Loh, G., and Blaut, M. Human intestinal microbiota: characterization of a simplified and stable gnotobiotic rat model. Gut Microbes. 2011, 2, 25-33; Fei, N. and Zhao, L. An opportunistic pathogen isolated from the gut of an obese human causes obesity in germfree mice. ISME. J 2013, 7, 880-884). Interestingly, the Parabacteroides, Bacteroides, and Akkermansia genus also dramatically rose in number in the garcinol-fed mice. Parabacteroides and Bacteroides belong to the Bacteroidetes phylum, and Akkermansia belong to the Verrucomicrobia phylum; this explains why the F/B ratio behaved as it did following induction by garcinol treatment. In the heatmap, we observed that Anaerobranca zavarzinii, Blautia coccoides, and Butyrivibrio proteoclasticus communities rose in number after HFD feeding, however garcinol administration not only lower those bacteria, but also Oscillospira eae, Mucispirillum schaedleri, Anaeroruncus colihominis, and Lachnospira pectinoschiza. In addition, garcinol increased the numbers of Akkermansia muciniphila, Bacteroides stercorirosoris, and Bacteroides xylanisolvens, which was diminished in the ND and HFD groups.

Anaerobranca zavarzinii, Blautia coccoides, and Butyrivibrio proteoclasticus belong to the Firmicutes phylum; Anaerobranca zavarzinii is positively correlated with IBD patients, and Blautia coccoides was increased in HFD-induced mice model. Butyrivibrio proteoclasticus is extremely sensitive to the toxic effects of unsaturated fatty acids and associated with obesity. On the other hand, Bacteroides stercorirosoris and Bacterides xylanisolvens belong to the Bacteroidetes phylum, and Akkermansia muciniphila to the Verrucomicrobia phylum. Andoh et al. (Andoh, A., Nishida, A., Takahashi, K., Inatomi, O. et al., Comparison of the gut microbial community between obese and lean peoples using 16S gene sequencing in a Japanese population. J Clin. Biochem. Nutr 2016, 59, 65-70) performed 16S rRNA sequence analysis of the gut microbiota profiles of obese and lean Japanese populations, and they found that Bacteroides stercorirosoris exists in lean Japanese people. Liu et al. (Liu, R., Hong, J., Xu, X., Feng, Q. et al., Gut microbiome and serum metabolome alterations in obesity and after weight-loss intervention. Nat. Med 2017, 23, 859-868) performed a metagenome-wide association study and serum metabolomics profiling in lean and obese, young, Chinese individuals. They linked intestinal microbiota alterations with circulating amino acids and obesity, and indicated that Bacteroides xylanisolvens was significantly enriched in lean controls.

Several studies have highlighted the effects of the mucin-degrading bacterium, Akkermansia muciniphila, which is more abundant in the mucosa of healthy subjects than in that of diabetic patients or animals (Liou, A. P., Paziuk, M., Luevano, J. M., Jr., Machineni, S. et al., Conserved shifts in the gut microbiota due to gastric bypass reduce host weight and adiposity. Sci Transl. Med 2013, 5, 178ra41). Many studies have demonstrated the dietary effect of Akkermansia muciniphila and how it also inhibits obesity. Dietary supplementation of an HFD with grape polyphenols resulted in dramatic changes in the gut microbial community structure, including a reduction in the F/B ratio and a bloom of Akkermansia muciniphila (Roopchand, D. E., Carmody, R. N., Kuhn, P., Moskal, K. et al., Dietary Polyphenols Promote Growth of the Gut Bacterium Akkermansia muciniphila and Attenuate High-Fat Diet-Induced Metabolic Syndrome. Diabetes 2015, 64, 2847-2858). All these studies support the suggestion that Akkermansia muciniphila has a potential role as a probiotic with anti-obesity effects, therefore we suggest that garcinol exhibits the prebiotic role.

Garcinol Treatment Increased the Number of Akkermansia Spp. and Affected AMPK Signaling Pathway by Inducing Endocannabinoid Expression

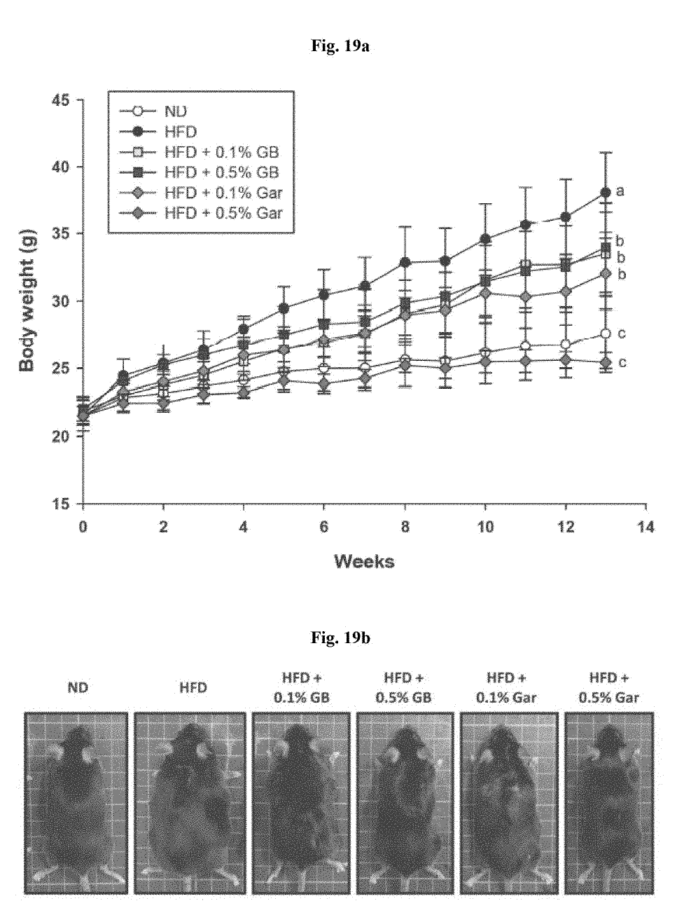

We further investigated the molecular mechanisms by which garcinol exerts anti-obesity effects. The protein levels of AMPK, p-AMPK, PPARγ, preadipocyte factor 1 (Pref-1), and SREBP-1 in HFD-fed C57BL/6 mice are shown in FIG. 19. HFD feeding resulted in decreased AMPK compared to that of the ND group, but it was increased by administration of low doses of garcinol (0.1%) in white adipose tissue. Interestingly, high dosages of garcinol (0.5%) did not elevate AMPK protein or p-AMPK protein levels. We estimated this might be associated with Akkermansia spp. Administration of A. muciniphila to HFD-induced obese mice for four weeks improved endocannabinoid content (Everard, A., Belzer, C., Geurts, L., Ouwerkerk, J. P. et al., Cross-talk between Akkermansia muciniphila and intestinal epithelium controls diet-induced obesity. Proc. Natl. Acad. Sci U.SA 2013, 110, 9066-9071) including 2-AG, 2-PG, and 2-OG. Within intestinal tissue, the increase of 2-AG reduces metabolic endotoxemia and systemic inflammation by increasing goblet cell and Treg populations. However, in perigonadal adipose tissue, the increase of 2-AG also enhanced the storing capacity of adipose tissue by stimulating preadipocyte differentiation (via upregulation of adipocyte PPARγ levels), and enhancing de novo fatty acid synthesis (via stimulation of lipoprotein lipase and upregulation of FAS levels and glucose uptake), diminishing fatty acid oxidation (via inhibition of AMPK), and enhancing triacylglycerol accumulation (via inhibition of lipolysis). 2-AG is a phospholipid-derived lipid containing an arachidonic acid chain within its chemical structure. 2-AG is also an intermediate in triacylglycerol and phospholipid metabolism, so mice treated with HFD can readily supply the substrate for 2-AG production. Pref-1 is identified as an inhibitor of adipocyte differentiation that is highly expressed in preadipocytes and that disappears during differentiation. Garcinol treatment caused an increased protein level of Pref-1 in epididymal adipose tissue which suggests garcinol may function in the maintenance of the preadipose state in HFD-fed mice.

Conclusion

The results revealed that garcinol treatment brought about an unexpected change in the composition of the gut microbiota in mice receiving a HFD, which may affect the underlying molecular mechanisms. Moreover, these findings reinforce the concept that changes in the gut microbial community, with the goal of increasing the Akkermansia population, can prevent obesity induced by HFD.

### Example 3: Comparative Evaluation of Garcinol and Composition Containing Garcinol, Pterostilbene and Anthocyanin for Weight Loss

The present invention studied the anti-obesity effects of garcinol compared to a composition comprising garcinol, pterostilbene and anthocyanin (garcinol blend (GB) in mammals. The study was conducted in vivo on 5 weeks old C57BL6 male mice. A total of 42 mice were involved in this study with 6 groups of 7 mice each. The groups were divided as in table 8.

The high fat diet (HFD) groups were fed 45% high fat diet for 16 weeks for the induction of obesity and concurrently administered the test substance as indicated in the aforesaid table. The normal group was fed with normal diet for 16 weeks.

TABLE 8
Study Groups

| | | Test substance | |
|---|---|---|---|
| Group | Diet | administered | |
| 1 | Normal Diet | None | |
| 2 | High Fat Diet (45%) | None | |
| 3 | High Fat Diet (45%) | 0.1% | GB |
| 4 | High Fat Diet (45%) | 0.5% | GB |
| 5 | High Fat Diet (45%) | 0.1% | Gar |
| 6 | High Fat Diet (45%) | 0.5% | Gar |

Body weight was monitored weekly, and the average body weight of each group (n=7) was expressed as the means±SE. The significance of difference among the six groups was analyzed by one way ANOVA and Duncan's multiple range tests. p<0.05, a, b, and c significantly different between each group.

The results indicated that Mice fed with HFD+0.5% Gar groups showed the most significantly decreased body weight and prevented weight gain compared to the HD fed group and HFD+GB group (FIGS. 19a and 19b). Mice administered with HFD+0.5% Gar showed the least weight gain compared to the other groups (Table 9) which is an unexpected finding and cannot be anticipated by a person skilled in the art.

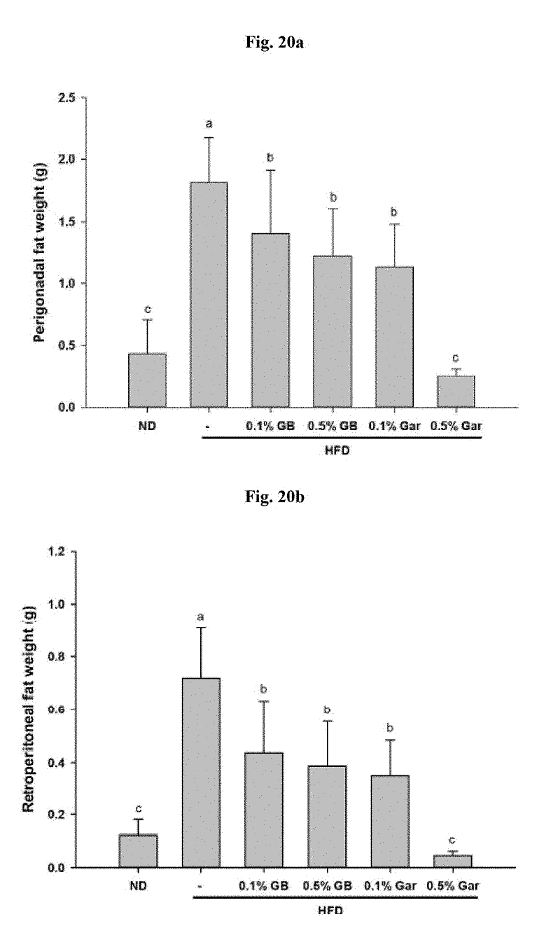

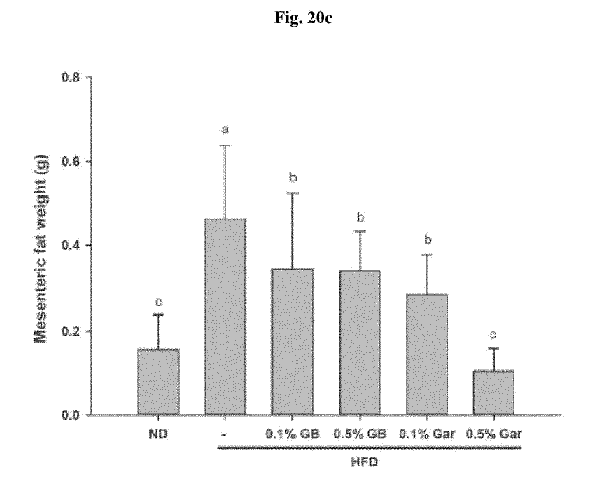

The effect of garcinol and garcinol blend on reducing the weight of perigonadal, retroperitoneal and mesenteric adipose tissues was also evaluated. The results indicated that 0.5% garcinol significantly reduced the weights of perigonadal, retroperitoneal and mesenteric adipose tissues compared to the garcinol blend (FIG. 20 a,b,c).

TABLE 9
Body weight of study animals administered with garcinol and garcinol blend

| | | | HFD + | HFD + | HFD + | HFD + |
|---|---|---|---|---|---|---|
| | ND | HFD | 0.1% GB | 0.5% GB | 0.1% gar | 0.5% gar |
| Initial | 21.5 ± 1.1^{a} | 21.6 ± 1.1^{a} | 21.9 ± 1.0^{a} | 22.1 ± 1.0^{a} | 2.1.5 ± 0.7^{a} | 21.5 ± 0.7^{a} |
| weight (g) | | | | | | |
| Final | 27.7 ± 2.7^{c} | 38.1 ± 3.0^{a} | 33.5 ± 3.1^{b} | 34.0 ± 3.3^{b} | 32.1 ± 2.6^{b} | 25.4 ± 0.8^{b} |
| weight (g) | | | | | | |
| Weight | 6.1 ± 1.8^{c} | 16.5 ± 2.7^{a} | 11.6 ± 2.8^{b} | 11.9 ± 2.6^{b} | 10.5 ± 2.1^{b} | 3.9 ± 0.6^{b} |
| gain (g) | | | | | | |

The average body weight of each group (n=7) is expressed as the mean±SE. The significance of difference among the six groups was analyzed by one way ANOVA and Duncan's multiple range tests. Value not sharing the same superscript letters in the same row are significantly different among group. p<0.05, a, b, and c significantly different between each group

Conclusion

Mice fed with HFD+0.5% garcinol showed significant reduction in weight compared to the garcinol blend. This is an unexpected finding and cannot be anticipated by a person skilled in the art.

From the abovementioned examples, it is evident that garcinol brings about inhibition of adipogenesis and promotes weight loss in a dose dependant manner compared to the garcinol blend containing pterostilbene and anthocyanin. Garcinol also modifies the gut microbiota and increases the viable colonies of beneficial microbe—Akkermansia muciniphila thereby maintain and improving general health and well being. The present invention confirms that garcinol is an effective anti-obesity molecule and can be effective administered as a stand alone or in combination with other weight loss ingredients for the management of obesity and related disorders.

While the invention has been described with reference to a preferred embodiment, it is to be clearly understood by those skilled in the art that the invention is not limited thereto. Rather, the scope of the invention is to be interpreted only in conjunction with the appended claims.

We claim:

1. A method for therapeutic management of obesity in mammals, said method comprising steps of administering effective concentration of a composition containing garcinol to said mammals to bring about a) inhibition of adipogenesis b) decrease in body weight, and visceral fat in said mammals.

2. The method as in claim 1, wherein the inhibition of adipogenesis is brought about by down regulation of genes selected from the group consisting of PPARγ, cEBPα, FAS, AP2, resistin and leptin.

3. The method as in claim 1, wherein inhibition of adipogenesis is brought about by up regulation of genes selected from the group consisting of p-AMPK, AMPK and PREF-1.

4. The method as in claim 1, wherein the visceral fat is selected from the group consisting of mesenteric fat, peritoneal fat and perigonadal fat.

5. A method of achieving energy balance in mammalian adipose cellular systems, said method comprising step of administering composition containing garcinol in effective amounts targeted towards mammalian pre-adipocytes and/or adipocytes to achieve effects of (a) increased inhibition of adipogenesis and (b) increased expression of secretory factors that function individually or in combination to specifically recruit brown adipocytes or brown like (beige or brite) adipocytes, c) induce brown like phenotype (beige or brite adipocytes) in white adipocyte depots, to bring about the effect of fat utilization and energy balance in said mammals.

6. The method as in claim 5, wherein the secretory factors include mitochondrial UCP-1, PRDM16, PGC-1α and BMP7.

7. A method of modifying the gut microbiota in mammals, said method comprising step of administering effective amounts of a composition containing garcinol to said mammals to bring about change in the gut microbiota.

8. The method as in claim 7, wherein the gut microbiota is selected from the Phylum Deferribacteres, Proteobacteria, Bacteroidetes, Verrucomicrobia and Firmicutes.

9. The method as in claim 7, wherein the gut microbiota is selected from the genus Lactobacillus, Butyrivibrio, Clostridium, Anaerobranca, Dysgonomonas, Johnsonella, Ruminococcus, Bacteroides, Oscillospira, Parabacterroides, Akkermanisa, and Blautia.

10. The method as in claim 7, wherein the gut microbiota is selected from the group consisting of Parabacteroides goldsteinii, Bacteroides caccae, Johnsonella ignava, Blautia wexlerae, Dysgonomonas wimpennyi, Blautia hansenni, Anaerobranca zavarzinni, Oscillospira eae, Mucispirillus schaedleri, Blautia coccoides, Anaerotruncus colihominis, Butyrivibro proteoclasticus, Akkermansia muciniphila, Lachnospora pectinoschiza, Pedobacter kwangyangensis, Alkaliphilus crotonatoxidans, lactobacillus salivarius, Anaerivibria lipolyticus, Rhodothermus clarus, Bacteroides stercorirosoris, Ruminocococcus flavefaciens, Bacteroides xylanisolvens, Ruminococcus gnavus, Clostridium termitidis, Clostridium alkalicellulosi, Emticicia oligoraphica, Pseudobutyrivibro xylanivorans, Actinomyces naturae, Peptoniphilus coxii, and Dolichospermum curvum.

11. The method as in claim 7, wherein the modification of gut microbiota is effective in therapeutic management of diseases selected from the group consisting of obesity, cardiovascular complications, Inflammatory bowel disease, Crohn's disease, Celiac disease, metabolic syndrome, liver diseases and neurological disorders.

12. A method for increasing the viable counts of Akkermansia muciniphila in the gut of mammals, said method comprising steps of administering effective amounts of a composition containing garcinol to mammals to bring about an increase in the colonies of said bacteria.

13. The method as in claim 12, wherein the increase in the colony counts of Akkermansia muciniphila reduces body weight through the AMPK signaling pathway by causing endocannabinoid release.

14. A method of therapeutic management of hyperlipidemia in mammals, said method comprising step of administering an effective concentration of a composition containing garcinol to bring about the effects of (i) reducing the amount of total blood cholesterol levels; (ii) reducing the concentrations of low density lipoproteins (LDL) and very low density lipoproteins (VLDL); (iii) increasing the concentrations of high density lipoproteins (HDL) and (iv) reducing concentrations of serum triglycerides, in the blood of said mammals.

15. The method as in claim 14, the medical cause of hyperlipidemia is obesity.

16. A composition containing garcinol for use as a prebiotic agent.

17. The composition as in claim 16, wherein the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.


SEQUENCE LISTING (18 sequences)

SEQ ID NO: 1 - DNA, 21 bp, Artificial Sequence
  Synthesised forward primer of mUCP1
aggcttccagtaccattaggt

SEQ ID NO: 2 - DNA, 22 bp, Artificial Sequence
  Synthesised reverse primer of mUCP1
ctgagtgaggcaaagctgattt

SEQ ID NO: 3 - DNA, 19 bp, Artificial Sequence
  Synthesised forward primer of mpgc1a
ccctgccattgttaagacc

SEQ ID NO: 4 - DNA, 19 bp, Artificial Sequence
  Synthesised reverse primer of mpgc1a
tgctgctgttcctgttttc

SEQ ID NO: 5 - DNA, 20 bp, Artificial Sequence
  Synthesised forward primer of mprdm16
tcccaccagacttcgagcta

SEQ ID NO: 6 - DNA, 20 bp, Artificial Sequence
  Synthesised reverse primer of mprdm16
atctcccatccaaagtcggc

SEQ ID NO: 7 - DNA, 21 bp, Artificial Sequence
  Synthesised forward primer of mBMP7
gagggctggttggtgtttgat

SEQ ID NO: 8 - DNA, 21 bp, Artificial Sequence
  Synthesised reverse primer of mBMP7
gttgcttgttctggggtccat

SEQ ID NO: 9 - DNA, 20 bp, Artificial Sequence
  Synthesised forward primer of m beta-actin
gaagtccctcaccctcccaa

SEQ ID NO: 10 - DNA, 16 bp, Artificial Sequence
  Synthesised reverse primer of m beta-actin
ggcatggacgcgacca

SEQ ID NO: 11 - DNA, 20 bp, Artificial Sequence
  Synthesised forward primer of m PPAR g
tcgctgatgcactgcctatg

SEQ ID NO: 12 - DNA, 21 bp, Artificial Sequence
  Synthesised reverse primer of m PPAR g
gagaggtccacagagctgatt

SEQ ID NO: 13 - DNA, 21 bp, Artificial Sequence
  Synthesised forward primer of m c EBP a
caagaacagcaacgagtaccg

SEQ ID NO: 14 - DNA, 21 bp, Artificial Sequence
  Synthesised reverse primer of m c EBP a
gtcactggtcaactccagcac

SEQ ID NO: 15 - DNA, 22 bp, Artificial Sequence
  Synthesised forward primer of m FAS
ctgagatcccagcacttcttga

SEQ ID NO: 16 - DNA, 19 bp, Artificial Sequence
  Synthesised reverse primer of m FAS
gcctccgaagccaaatgag

SEQ ID NO: 17 - DNA, 18 bp, Artificial Sequence
  Synthesised forward primer of m AP2
catggccaagcccaacat

SEQ ID NO: 18 - DNA, 20 bp, Artificial Sequence
  Synthesised reverse primer of m AP2
cgcccagtttgaaggaaatc